(19) United States
(12) Patent Application Publication
(10) Pub. No.: US 2023/0225940 A1
(43) Pub. Date: Jul. 20, 2023

(54) BREASTMILK SAMPLE COLLECTION

(71) Applicant: Anue AS, Bodo (NO)

(72) Inventors: Jeanette Therese Gronnslett, Bodo (NO); Goran Jensen Borkamo, Bodo (NO); Anne Lise Oiesvold, Bodo (NO)

(21) Appl. No.: 18/019,014

(22) PCT Filed: Jul. 30, 2021
(86) PCT No.: PCT/EP2021/071503

§ 371 Date: Jan. 31, 2023

(30) Foreign Application Priority Data
Jul. 31, 2020 (GB) 2011998.8

(51) Int. Cl. A61J 1/12; A61M 1/06; G01N 1/18

(52) U.S. Cl. CPC A61J 1/12; A61M 1/06; G01N 1/18

(57) ABSTRACT

A system for collecting a sample of a liquid, the system including a liquid storage vessel including an opening, and a capping element configured to seal the opening of the storage vessel is provided. The capping element includes a chamber configured to store a sample of the liquid separate to the liquid storage vessel. The capping element includes a pipette element defining the chamber that is configured to store the sample of liquid taken from the storage vessel.

This application is entitled to the benefit of, and incorporates by reference essential subject matter disclosed in PCT Application No. PCT/EP2021/071503 filed on Jul. 30, 2021, which claims priority to GB Patent Appln. No. 2011998.8 filed Jul. 31, 2020, which are hereby incorporated by reference in their entireties.

### BACKGROUND OF THE INVENTION

### 1. Technical Field

The present invention relates to the collection and transport of donor breastmilk samples, e.g. for testing in medical or research settings or other use (e.g. as eye drops). However, it is also possible for the invention to be applied to the collection and transport of samples of other foodstuff liquids or even various liquids across a range of fields, such as samples of adhesives, paints or other chemicals.

### 2. Background Information

Current methods for the storage and transport of breastmilk involve the use of simple containers such as plastic pouches, bottles, or zip lock bags. Typical state of the art products have numerous disadvantages with regards to long-term storage, transport and testing. These disadvantages are particularly apparent in the field of donor breastmilk, provided for e.g. babies born prematurely. Donor breastmilk is typically collected at the home of the donor, where it is frozen for longer-term storage (i.e. to prevent spoilage), or for later transportation (e.g. to a donor milk bank or hospital). Typically the breastmilk must be thawed at the hospital before a sample can be taken for testing. However, during collection and testing, the breastmilk is susceptible to contamination. When the breastmilk is being transported, temperature fluctuations can reduce its nutritional content.

As a result, current methods are lacking when it comes to collection and transportation of frozen breastmilk, which may thaw slightly during transit, and which can be subsequently refrozen at the hospital or milk bank. When the breastmilk is eventually tested, a large amount if not all of the collected breastmilk must be thawed for testing in order for a sample to be taken that is representative. The rest of the breastmilk in the container must be used quickly after it has been thawed, but the portions used to feed newborn babies are very small. This can lead to significant amounts of wastage. The applicant has realized that an improved process for storage, transportation and testing of breastmilk donations could result in significantly reduced wastage of breastmilk. Many of the same considerations may apply when taking a sample of other liquids for testing purposes, e.g. where the lifetime of the liquid is impacted by opening a container to take a sample.

The present disclosure seeks to provide improved systems and methods for collecting a sample of breastmilk or other liquid.

### SUMMARY

From a first aspect, the invention provides a system for collecting a sample of a liquid, the system comprising: a liquid storage vessel comprising an opening; and a capping element; wherein the capping element is configured to seal the opening of the storage vessel; and wherein the capping element comprises a chamber configured to store a sample of the liquid separate to the liquid storage vessel.

In the system according to the first aspect of the invention, a sample of liquid can be collected from a storage vessel with a low probability of contamination from outside sources. Collecting and storing a sample from a larger storage vessel within a chamber of a capping element for the vessel allows the sample to be separated without the need for decanting into additional vessels, or the use of separate collection equipment, reducing the likelihood of contamination of the sample.

What is meant by the sample being stored separate to the liquid storage vessel is that the chamber holds the sample physically apart from the rest of the liquid in the storage vessel. Once the liquid sample is collected within the chamber of the capping element it is no longer in fluid contact with the liquid in the storage vessel, and is held such that it does not flow back into the storage vessel. This means that the sample can be independently taken for testing by removing the chamber. As will be explained below, this may occur before or after freezing the system. It will further be understood that a sample may have a predefined volume, or volume range, that is typically a lot smaller than the volume of the liquid storage vessel. The chamber may be configured to define the volume or volume range of the sample that is stored in the capping element. In at least some embodiments the chamber may have an internal volume of up to 20 ml, but in preferred embodiments the chamber has an internal volume of 2-5 ml. For example, the chamber may have an internal volume of about 2 ml or 3 ml for collecting a breastmilk sample, as this is the sample size typically used for bacterial testing or nutritional testing of breastmilk.

In a set of embodiments the capping element comprises a capping part configured to seal the opening of the liquid storage vessel and the chamber is separable from the capping part. The chamber may be separated from the capping part by twisting, pulling, or other mechanical manipulation. In some examples, the chamber may have a frangible connection to the capping part. In some examples, the chamber may have a separable connection to the capping part that comprises a screw fitting, bayonet fitting, snap-fit, etc. Including a chamber separable from the capping part allows the liquid stored within the storage vessel to remain sealed from the outside environment (by the capping part) when the chamber containing the liquid sample is removed.

The connection between the capping part and the chamber is preferably arranged such that the chamber may be easily detached from the capping part. This may be beneficial in applications in which the liquid storage vessel is subjected to freezing temperatures (e.g. down to −30° C.). In such applications, the liquid storage vessel may be frozen with the capping part sealing the liquid storage vessel, and the chamber sealing the capping part, and hence it is advantageous if the chamber may be easily removed even when both the capping part and the liquid storage vessel are frozen.

In some embodiments the capping part comprises a first liquid-conveying connector that is uncovered when the chamber is separated from the capping part. The inclusion of a connector in the capping part allows the storage vessel to be connected to other equipment via the capping part after the sample has been collected and the chamber removed. For example, the storage vessel may be connected to a fluid transfer line or syringe for clinical usage.

In some embodiments the chamber comprises a second liquid-conveying connector configured to mate with the first liquid-conveying connector. This may allow the chamber to be reliably connected and disconnected from the capping part. For example this may allow the chamber to be used to collect multiple samples in succession.

In some embodiments, the first connector (and optionally the second connector) are medical connector parts. In a further set of embodiments, the first connector and/or second connector conforms to the requirements of one of the ISO 80369 series of small-bore connector standards. The aim of this series of standards is to prevent misconnections between fluid transfer lines for different clinical uses, e.g. between enteral feeding tubes and IV lines. ISO 80369-1:2010 specifies the health fields in which liquid-conveying connectors are intended to be used. These healthcare fields of use include, but are not limited to, applications for: breathing systems and driving gases; enteral and gastric; urethral and urinary; limb cuff inflation; neuraxial devices; intravascular or hypodermic. In some embodiments, the first liquid-conveying connector and/or the second liquid-conveying connector comprise an ENFit connector or any other enteral connector compliant with ISO 80369-3. Preferably the first connector comprises a female ENFit connector, and the second connector comprises a male ENFit connector. The Applicant has recognised that providing connectors which conform to ISO 80369-3 (ENFit) may help to prevent misconnection of the connectors, as well as preventing misconnection to other devices. For example, it may prevent a Luer fit syringe from being connected to the ENFit connector part so that only enteral feeding syringes can be connected to the capping part. This may be particularly beneficial for the collection of breastmilk, as it allows the chamber and/or the capping part to be correctly connected to other equipment for enteral feeding.

In some examples in which the first liquid conveying connector and the second liquid-conveying connector comprise ENFit connectors, the first liquid conveying connector and the second liquid-conveying connector may comprise threading in the form of a partial thread or full thread, in order to effectively secure the connection between the chamber and the capping part. In some examples the first liquid conveying connector and/or the second liquid-conveying connector may comprise a Nutrisafe or Nutrisafe 2 compatible connector or any other medical standard enteral feeding connector.

In some embodiments, the system further comprises a plug; wherein the plug comprises two ends: a first end configured to seal the chamber of the capping element, and a second end configured to seal the capping part, after the chamber is separated from the capping part. The plug having two ends configured to seal the chamber and the capping element, respectively, advantageously may allow a single type of plug to be manufactured that can be used to seal the chamber or the capping element. This may reduce the cost of manufacturing by allowing a single mold to be used for plugs for use with both the chamber and the capping part. Preferably the system includes at least one plug for sealing the capping part after the chamber has been separated, to prolong the lifetime of the liquid in the storage vessel after the sample has been removed. In some embodiments the system may include at least two plugs, i.e. a first plug for sealing the capping part (using the second end) and a second plug for sealing the chamber (using the first end). The plug can therefore be used to keep the chamber sealed while it is being further transported or waiting for testing of the sample. However, it should be appreciated that a plug may not always be necessary to seal either the capping part or the chamber, as the system may be frozen quickly after collecting the liquid (e.g. fresh breastmilk) and the openings concerned may be small enough that contamination is considered low risk.

In a potentially overlapping set of embodiments, the opening of the liquid storage vessel comprises a threaded interface. Such a threaded interface may be useful to connect the storage vessel to other components directly. For example the threaded interface may be connected to a breast pump (optionally via a disposable breast shield) to allow for breast milk to be collected directly within the storage vessel, without requiring the use of additional containers, such as the collection bottle of a breast pump, reducing the risk of contamination of the breastmilk. The threaded interface may be broken or discontinuous, or may be a single continuous thread.

The liquid storage vessel can take any suitable form as a container for storing liquid. In various embodiments, the liquid storage vessel comprises a pouch (e.g. made of a flexible material, e.g. such as polyethylene) or bottle (e.g. made of a rigid or semi-rigid material, e.g. such as polypropylene). The liquid storage vessel can define any suitable volume based on the liquid being stored therein. In various embodiments, in addition or alternatively, the liquid storage vessel has an internal volume of at least 30 ml, 40 ml, 50 ml, 60 ml, 70 ml, 80 ml, 90 ml, 100 ml, 110 ml, 120 ml, 130 ml, 140 ml, or 150 ml. In various embodiments, in addition or alternatively, the liquid storage vessel has an internal volume of up to 200 ml, 210 ml, 220 ml, 230 ml, 240 ml, 250 ml, 260 ml, 270 ml, 280 ml, 290 ml or 300 ml. Liquid storage volumes between 30 ml and 300 ml may be particularly appropriate when breastmilk is being expressed and collected in the liquid storage vessel.

In a first set of embodiments the capping element comprises a pipette element defining the chamber configured to store the sample of liquid taken from the storage vessel. The pipette element may allow a sample to be collected from the liquid storage vessel through the creation of a partial vacuum within the pipette element, and a sample may advantageously be held within the pipette element by surface tension of the liquid. The sample being stored in a pipette element may allow for a user to easily squeeze out a few drops of breastmilk as baby eye drops, e.g. before collecting another sample for milk bank testing or even as an alternative.

It will be understood that a pipette element functions like a pipette by drawing in the liquid sample. In order to draw in the sample, the pipette element may be at least partially flexible such that a user can squeeze the pipette element to create a partial vacuum that pulls liquid into the chamber from the storage vessel. The pipette element may comprise a flexible bulb connected to a liquid tube defining the chamber, as is conventional. However, it has been appreciated that a conventional pipette is designed to draw in and measure different sample sizes, whereas it is desirable for the pipette element in embodiments of the present invention to draw in a fixed sample volume upon operation. Thus a liquid tube (which would be marked with graduations in a conventional pipette) is not necessary. In at least some embodiments, the pipette element comprises a flexible bulb defining the chamber. This means that the sample is pulled directly into the flexible bulb to be stored in the chamber. As mentioned above, the chamber i.e. flexible bulb may be configured to define the volume (or volume range) of the sample. In at least some embodiments, the flexible bulb is at least partially transparent to allow a user to watch the liquid being drawn into the chamber. In at least some embodiments, the flexible bulb is made from a flexible elastomeric material, e.g. thermoplastic elastomers such as silicone. Preferably the material(s) chosen for the pipette element is able to withstand freezing temperatures e.g. down to −30° C.

An advantage of a pipette element is that the liquid sample is held in the chamber by a vacuum effect. Even if the pipette element is separated from the liquid storage vessel with the liquid sample in an unfrozen state, the sample will not fall out (unless the pipette element is squeezed). This means that the pipette element does not need a one-way valve or cap/plug of its own. However, a plug as described above may optionally be used to seal the pipette element, for example to avoid contamination of the sample stored inside.

In some embodiments, in addition or alternatively, the pipette element comprises a flat base portion configured to support the pipette element in an upright standing position. In those embodiments comprising a flexible bulb, the flexible bulb may be shaped to have such a flat base portion. Unlike a normal pipette, the flat base portion allows for the pipette element to be turned upside down and stand by itself in an upright position. This can make it easier to insert a test strip or probe into the chamber to test the liquid sample.

Although the pipette element may comprise a flat base portion, it should be recognized that the base portion may take any form that allows the pipette to be turned upside down and stand by itself in an upright position such that the pipette element is self-supporting. Hence the pipette element may, in some examples, comprise a base portion of an alternative shape that allows the pipette element to be turned upside down and stand by itself in an upright position. The base portion of the pipette element may comprise one or more supporting projections that allow it to stand by itself in an upright position. For example, the base portion may comprise two or more projections from its surface, e.g. three projections, forming a tripod beneath the base portion of the pipette element, allowing the pipette element to be self-supporting. Thus, in some examples, the pipette element may comprise a base portion configured such that the pipette element is self-supporting.

In some examples, the relative dimensions of the pipette element are selected in order to facilitate freezing of a liquid sample, and its subsequent storage within the pipette element. Thus in some examples the opening of the pipette element defined by the liquid tube may be narrow relative to the width of the flexible bulb of the pipette element and/or the base portion of the pipette element. The width of the opening of the pipette element may be dependent on the properties of the liquid to be stored in the pipette element. The width of the opening of the pipette element may be dependent on the surface tension of the liquid to be held within the pipette element in order to allow the liquid sample to be held within the pipette element by surface tension of the liquid. For example if the pipette element is used to store a liquid with high surface tension, the width of the opening of the pipette element may be greater than if the pipette element is used to store a liquid having lower surface tension. In some examples, the width of the opening of the pipette element may be no more than 8 mm or no more than 6 mm. In some examples the width of the opening of the pipette element may be between 4 and 7 mm, preferably between 5 mm and 6 mm.

The width of the flexible bulb of the pipette element may be dependent on the desired volume of the liquid sample to be stored in the pipette element. In some examples, the width of the flexible bulb may be at least 15 mm, at least 20 mm, or at least 25 mm. The width of the flexible bulb may be no more than 60 mm, no more than 50 mm, or no more than 40 mm, but is preferably no more than 30 mm. However it will be appreciated that in some examples, the width of the flexible bulb may be significantly greater than this.

The width of the base portion of the pipette element may be dependent on the desired volume of the liquid sample to be stored in the pipette element while allowing the pipette element to be turned upside down and stand by itself in an upright position. In some examples, the width of the base portion of the pipette element may be at least 5 mm, at least 10 mm, or at least 15 mm. The width of the base portion of the pipette element may be no more than 50 mm, no more than 40 mm, no more than 30 mm, or no more than 20 mm. However it will be appreciated that in some examples, the width of the base portion of the pipette element may be significantly greater than this. In some examples the flexible bulb of the pipette element may be non-circular and in these cases the above dimensions refer to the maximum width.

In one preferred example for collecting a breastmilk sample, the width of the opening of the pipette element is 5.5 mm, the maximum width of the base portion of the pipette element is 13.9 mm, and the maximum width of the flexible bulb of the pipette element is 24.3 mm.

In embodiments wherein the chamber comprises a second liquid-conveying connector, the pipette element may be arranged to have the liquid-conveying connector at an open end and the flat base portion at an opposite end. This means that the pipette element can rest upright on the flat base portion with the liquid-conveying connector facing upwards. This makes it easier to handle the pipette element, which can be placed down on a work surface, and to access the sample through the liquid-conveying connector.

In some embodiments, the capping element comprises a dual pipette element defining two pipette chambers, wherein each pipette chamber is configured to store some of the sample of liquid taken from the storage vessel. The use of a dual pipette element allows two separate samples of liquid to be collected, which may be used for two different purposes after collection. For example, in the collection of breastmilk, a first sample may be used for bacteriological testing, and a second sample may be used for testing the nutritional contents of the breastmilk, or one of the samples may be used as baby eye drops.

In some embodiments the two pipette chambers are separable from one another. This may facilitate separate uses for the samples collected within the two pipette chambers, for example allowing the two samples to be tested independently, or allowing a first sample to be stored while a second sample is tested, or allowing for one sample to be used as baby eye drops.

In a second set of embodiments the capping element comprises a syringe; wherein the syringe comprises a barrel, and wherein the barrel of the syringe defines the chamber configured to store the sample of the liquid separate to the storage vessel. The capping element comprising a syringe may allow a liquid sample of a specific volume to be collected more accurately. The sample being stored in a syringe may also make it easier to handle the sample for testing or otherwise administer the sample e.g. as baby eye drops.

In a third set of embodiments the chamber of the capping element comprises an aperture; wherein the chamber is rotatable around a central axis of the opening between an open position in which the aperture is in fluid communication with the opening of the storage vessel, and a closed position in which there is no fluid communication between the aperture and the opening of the storage vessel.

According to a second aspect, the invention provides a method of collecting a sample of liquid from a storage vessel, the method comprising: at least partially filling the storage vessel with a liquid; sealing the storage vessel with a capping element comprising a chamber; manipulating the storage vessel and/or the capping element such that a sample of the liquid flows from the storage vessel into the chamber of the capping element; and storing the sample of liquid in the chamber of the capping element.

It will be appreciated that this is a convenient method of collecting liquid in a storage vessel while also taking a sample of the liquid which is stored separately in the chamber of the capping element and may therefore be taken independently of the storage vessel. The chamber may be removed as soon as the vessel has been filled or at a later time. Thus in some examples. The method may further comprise removing the chamber containing the sample of liquid.

In some embodiments the liquid is breastmilk. The method may optionally further comprise: connecting a breast pump to the storage vessel and operating the breast pump to introduce breastmilk into the storage vessel.

In some potentially overlapping embodiments the method further comprises the steps of: freezing the storage vessel and the capping element containing the sample of liquid; and removing the chamber containing the frozen sample. In this way the combination of storage vessel and capping element may be frozen together, and the chamber containing a sample of liquid may be subsequently thawed. This advantageously allows for a small volume of liquid to be thawed for testing without requiring the entire volume of liquid stored contained in the storage vessel to be thawed. A representative sample of the liquid may then be tested or analyzed while avoiding unnecessary temperature fluctuation of the larger volume of liquid. If the storage vessel has been intermittently thawed, going from a frozen state via a semi-thawed state and back to a thawed state (e.g. during transportation to a milk bank), the small separate sample in the capping element would likely thaw more or less completely, prior to the larger volume in the storage vessel. Such thawing and re-freezing can be a source for bacterial growth and, as such, if an analyzed sample from the capping element is non-contaminated with bacterial growth, it may be assumed that the main volume in the storage vessel is either of the same quality or even less contaminated than the analyzed sample. In other words, the sample taken for analysis may actually be assumed to represent a lowest possible quality for the liquid in the storage vessel.

It will be appreciated that removing the chamber may comprise removing the chamber and leaving a part of the capping element attached to the storage vessel, e.g. a capping part as described above, or may comprise removing the entire capping element, and (optionally) subsequently sealing the opening of the storage vessel. In some alternative embodiments the chamber storing the liquid sample may be removed before freezing the storage vessel. As mentioned above, the capping element may comprise a pipette element defining the chamber and hence the sample taken from the storage vessel is held inside the chamber by a vacuum effect in its liquid state.

Thus, in some examples, removing the chamber may comprise removing the capping element from the storage vessel, and optionally resealing the storage vessel.

In some examples, the capping element comprises a capping part arranged between the storage vessel and the chamber, the capping part being separable from the chamber. In such embodiments, removing the chamber may comprise separating the chamber from the capping part. In some such examples, it may be beneficial to seal the capping part and/or the chamber in order to prevent contamination of their contents. Thus in some such examples the method may further comprise sealing the capping part and/or the chamber. For example, the capping part and/or the chamber may be sealed using a plug as described above.

According to a third aspect, the invention provides a system for collecting a sample of a liquid, the system comprising: a liquid storage vessel comprising a first compartment and a second compartment connected by a channel, wherein the channel is arranged for liquid to flow from the first compartment to the second compartment in order to store a sample of the liquid in the second compartment separate to the first compartment; and wherein the first compartment and the second compartment are different sizes.

In a system according to the third aspect of the invention, a sample of liquid can be collected in a second compartment of the liquid storage vessel separately to the first compartment. As the two compartments are formed within the same vessel, a small sample may be isolated without the need for the liquid to be decanted into additional vessels, and without separate collection equipment being required, reducing the likelihood of contamination of the sample.

In some embodiments the second compartment comprises a pipette element. The pipette element allows a sample to be easily drawn into the second compartment by establishing a partial vacuum within the pipette element, where it may be held by the surface tension of the liquid between the pipette element and the channel.

In some embodiments the first compartment and the second compartment are made of a first material, and the channel is made of a second material. For example the first compartment and the second compartment of the liquid storage vessel may be made from polyethylene, while the channel may be made from polyvinylchloride (PVC) or polyurethane.

In some embodiments the first compartment further comprises an opening comprising a threaded interface. The opening may allow the first compartment to be at least partially filled with a liquid, while the threaded interface may allow the opening to be sealed using, for example, a cap with a corresponding thread. The threaded interface may conveniently be used to directly attach the liquid storage vessel to a breast pump.

According to yet another aspect, the invention provides a method of collecting a sample of a liquid from a storage vessel comprising a first compartment and a second compartment connected by a channel arranged for liquid to flow from the first compartment to the second compartment; the method comprising: at least partially filling the first compartment with a liquid; manipulating the storage vessel such that a sample of the liquid flows from the first compartment to the second compartment via the channel; and disconnecting the channel between the first compartment and the second compartment.

It will be appreciated that this is a convenient method of collecting liquid in a storage vessel while also taking a sample of the liquid which is stored separately in the second compartment. The channel between the first and second compartments may be disconnected in any suitable manner, for example, by sealing the channel closed, or by cutting or tearing to separate the first and/or second compartment from the channel.

In some embodiments the liquid is breastmilk. The method may optionally further comprise: connecting a breast pump to the storage vessel and operating the breast pump to introduce breastmilk into the first compartment.

In some embodiments, the method further comprises freezing the storage vessel with the sample of liquid stored in the second compartment before disconnecting the channel. This may facilitate the disconnection of the channel and reduce the likelihood of liquid being lost from the storage vessel during the disconnection process.

In some embodiments, the method further comprises removing the second compartment from the storage vessel. Removing the second compartment may allow the sample of liquid within the second compartment to be transported, for example for testing, without affecting the remaining liquid within the first compartment of the storage vessel, which may be required to be kept in a temperature controlled environment.

In some embodiments, the first compartment, the second compartment and the channel are made of a thermoplastic, and disconnecting the channel comprises applying a heat weld to the channel. Applying a heat weld, for example by using a hot bar welding or impulse welding process, allows opposite sides of the channel to be fused together by the application of heat to the thermoplastic material. In this way the sample stored in the second compartment may be effectively and permanently disconnected from the liquid in the first compartment.

### BRIEF DESCRIPTION OF THE DRAWINGS

Certain examples of this disclosure will now be described with reference to the accompanying drawings, in which:

FIGS. 1A-1C schematically illustrate a liquid sample collection system according to some embodiments of the invention;

FIG. 2 schematically illustrates a capping part and a pipette element according to some embodiments of the invention;

FIGS. 3A-3D schematically illustrate possible connectors of a pipette element according to some embodiments of the invention;

FIG. 4 schematically illustrates a dual pipette element according to some embodiments of the invention;

FIGS. 5A and 5B schematically illustrate possible connectors of a dual pipette element according to some embodiments of the invention;

FIG. 6 schematically illustrates the use of a breast pump and breast shield with a liquid storage vessel according to some embodiments of the invention;

FIG. 7 is a flow chart describing steps of an exemplary process of collecting a sample of a breastmilk using the liquid sample collection system according to some embodiments of the invention;

FIGS. 8A and 8B schematically illustrate a plug used to seal elements of a liquid sample collection system according to some embodiments of the invention;

FIGS. 9A and 9B schematically illustrate some alternative plugs used to seal a pipette element of a liquid sample collection system according to some further embodiments of the invention;

FIG. 10 is a schematic illustration of a liquid sample collection system according to some other embodiments of the invention;

FIGS. 11A and 11B are a schematic illustration of a liquid sample collection system according to some other embodiments of the invention;

FIG. 12 is a flow chart describing steps of an exemplary process of collecting a sample of a breastmilk using the liquid sample collection system according to FIGS. 11A and 11B;

FIG. 13 is a schematic illustration of a liquid sample collection system according to some embodiments of the invention;

FIG. 14 is a flow chart describing steps of an exemplary process of collecting a sample of a breastmilk using the liquid sample collection system according to FIG. 13;

FIGS. 15A and 15B schematically illustrate liquid sample collection systems according to some others embodiments of the invention; and

FIG. 16 is a schematic illustration of a liquid sample collection system according to some further embodiments of the invention.

### DETAILED DESCRIPTION

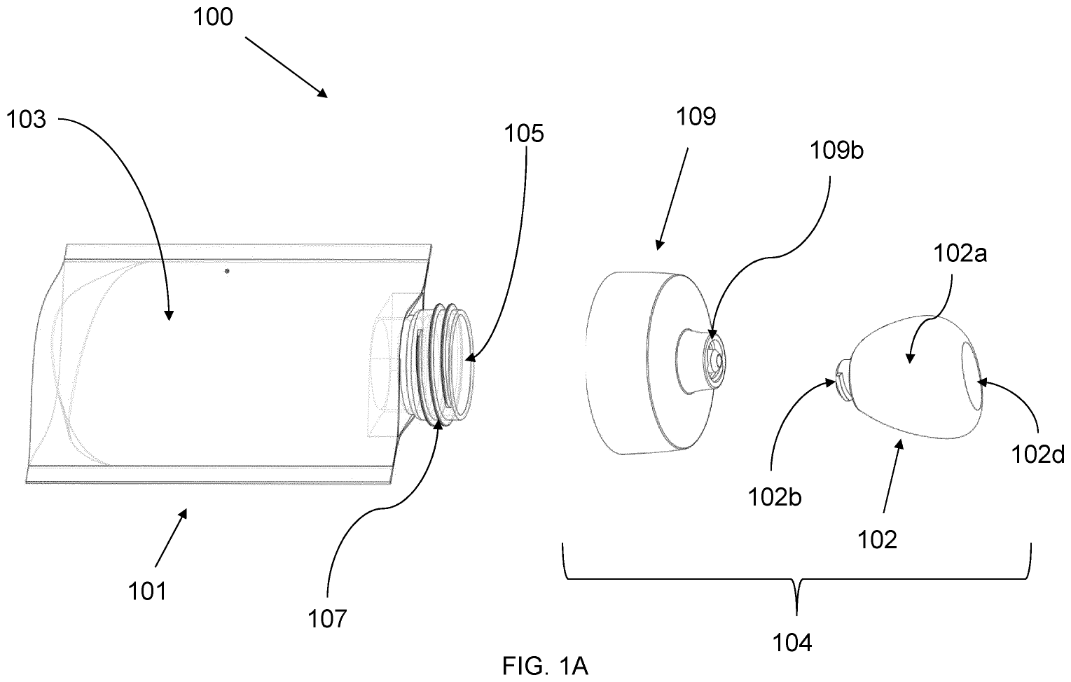

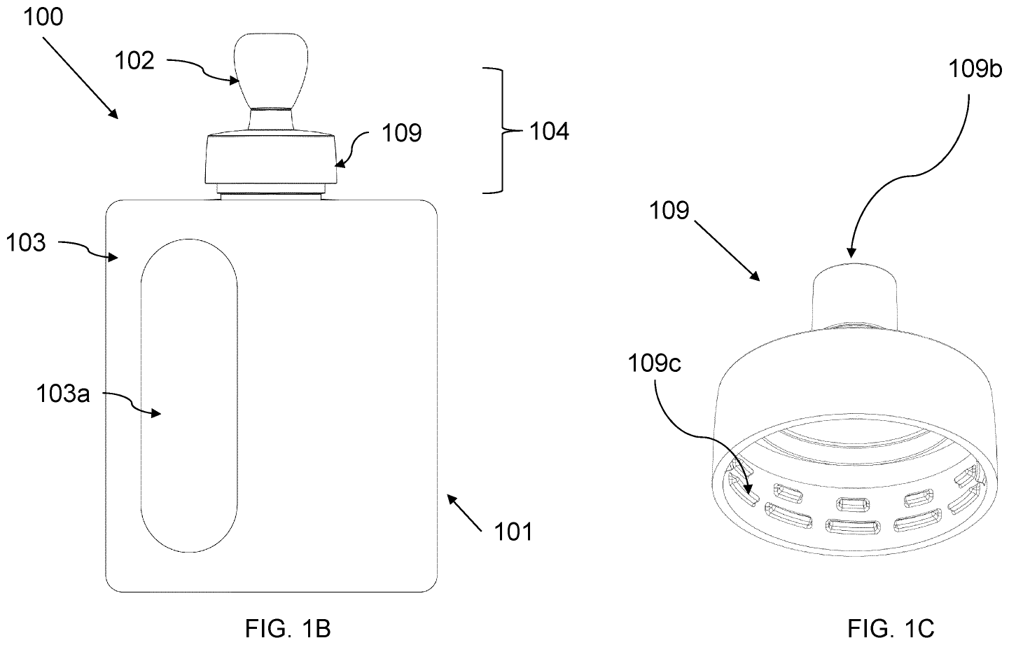

FIGS. 1A and 1B schematically illustrate a liquid sample collection system 100 according to a first embodiment of the invention. The liquid sample collection system 100 may be used to isolate a small sample of liquid from a larger volume in a liquid storage vessel, as may be required for testing or analysis purposes. The liquid sample collection system 100 may be used to collect a sample of liquid with a low probability of contamination from outside sources. For example, the liquid sample collection system 100 may be used to collect a small sample of breastmilk to allow the fat content and/or presence of contaminants to be tested.

In some embodiments, the liquid sample collection system 100 comprises a hollow storage vessel 101 and a capping element 104, which seals the storage vessel 101. The capping element comprises a pipette element 102, and a capping part 109. The pipette element 102 is made of a pliable material, such as silicone, and comprises a connector 102b, and a bulb chamber 102a. A sample of liquid may be separated from the liquid within the storage vessel 101 and stored within the chamber 102a of the bulb, as will be described in the following. The bulb chamber 102a of the pipette element 102 may have an internal volume of up to 20 ml, but in preferred embodiments has a volume of 2-5 ml, for example about 2 ml. The pipette element 102 is transparent or semi-transparent in order for samples of liquid collected within to be visible.

The storage vessel 101 comprises a pouch 103 with an opening 105, which comprises a screw thread interface 107 on its outer surface. The pouch 103 is preferably made of a flexible material. The pouch 103 may be made of a flexible plastic, such as polyethylene. The pouch 103 may be made of a BPA-free plastic. Alternatively the pouch 103 may be made of a metallic foil, waterproofed paper material, or any suitable composite or laminated material. In embodiments in which the pouch 103 is opaque, the pouch 103 may comprise a transparent window 103a, as shown in FIG. 1B, such that the contents of the pouch are visible from the exterior of the pouch. However the storage vessel 101 does not need to be a pouch 103 as shown and could instead be a bottle, for example a breastmilk collection bottle as is widely available from breast pump manufacturers, with the opening 105 being the bottle neck opening.

The capping part 109 of the capping element 104 may be any part that facilitates connection between the opening 105 of the storage vessel 101 and the pipette element 102. In the embodiment shown in FIGS. 1A and 1B, the capping part 109 comprises a threading 109c, illustrated in FIG. 1C, that is configured to mate with the screw thread interface 107 of the opening 105, such that the capping part 109 can be attached to the pouch 103 at the opening 105. The threading 109c of the capping part 109 may be continuous or discontinuous, as long as the function of mating with the corresponding threading of the screw thread interface 107 of the opening 105 is maintained. Preferably, the threading 109c of the capping part 109 is discontinuous as shown in FIG. 1C in order to reduce the cost of manufacture. When attached, the opening 105 is effectively sealed. The capping part 109 also comprises a liquid conveying connector 109b, configured to mate with a matching connector 102b of the pipette element 102. In this way the pipette element 102 can be attached to the capping part 109, sealing the storage vessel 101. The capping element 104 may be attached to the opening 105 as a single part, i.e. with the pipette element 102 already connected to the capping part 109.

Although the opening 105 of the embodiment shown in FIG. 1 comprises a screw thread interface 107, in some embodiments the opening 105 does not comprise a threading, and instead comprises clips on its outer surface. In other embodiments, the opening 105 may be integral with the pouch 103 itself. In some embodiments, no capping part is present, and the opening 105 of the pouch 103 itself comprises a liquid conveying connector 109b, or simply provides an opening which can be covered by the capping element without the need for mating connectors.

In this embodiment the connectors 109b, 102b conform to ISO 80369-3 (ENFit); the connector 109b comprises a female ENFit connector hub, and the connector 102b comprises a male ENFit connector. The Applicant has recognized that providing connectors which conform to ISO 80369-3 (ENFit) may help to prevent misconnection of the pipette element 102, as well as preventing misconnection to other devices. For example, it may prevent a Luer fit syringe from being connected to the ENFit connector 109b so that only enteral feeding syringes can be connected to the capping part 109. However, it will be appreciated that the connectors 109b, 102b in this embodiment, and in other embodiments described below, may conform to any other medical standard enteral feeding connector instead of ISO 80369-3. Some known enteral connector types that may be applied to the liquid sample collection systems described herein are ENFit, Nutrisafe and Nutrisafe 2.

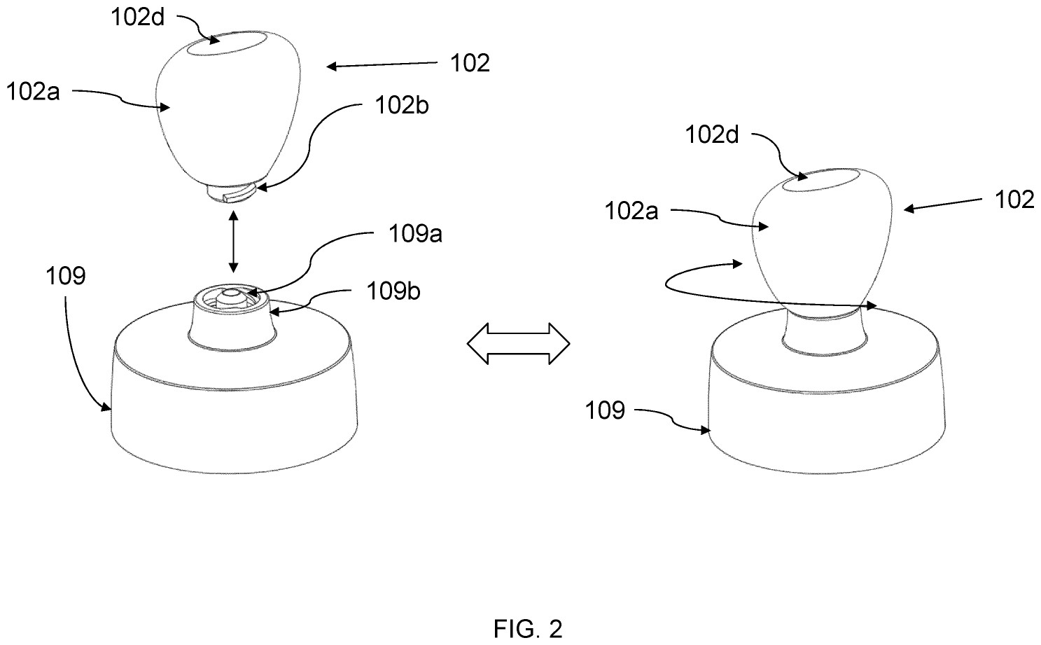

FIG. 2 shows how the pipette element 102 is attached and removed from the capping part 109 in embodiments in which the connectors 102b, 109b are ENFit connectors. To attach the pipette element 102, the male connector 102b is first inserted into the female connector 109b of the connection part 109, and the pipette element 102 is then twisted, locking the ENFit connector 102b with the ENFit connector 109b of the capping part 109 to form a seal. To remove the pipette element 102, this process is carried out in reverse.

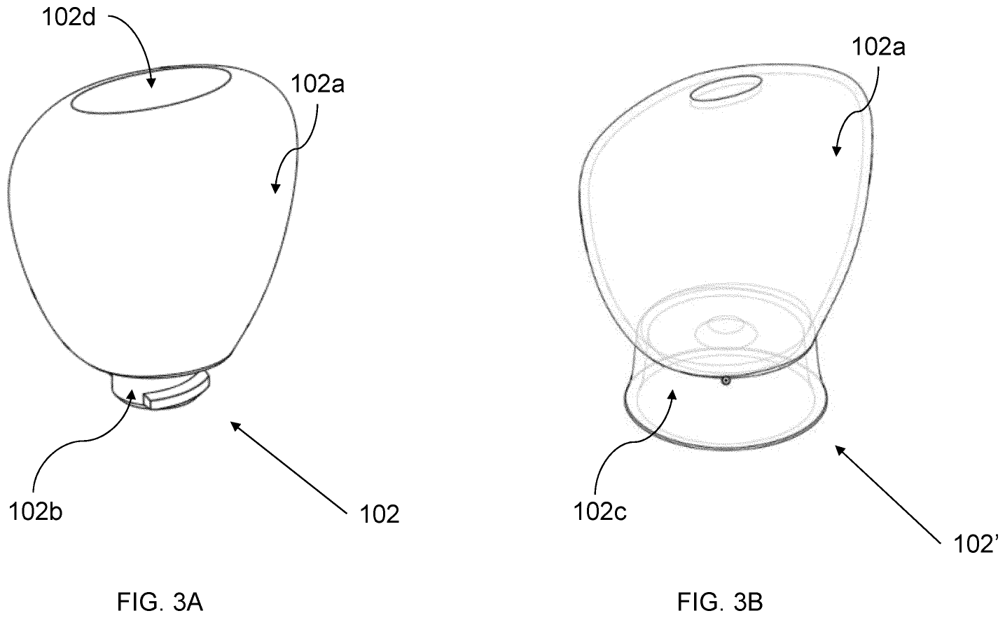

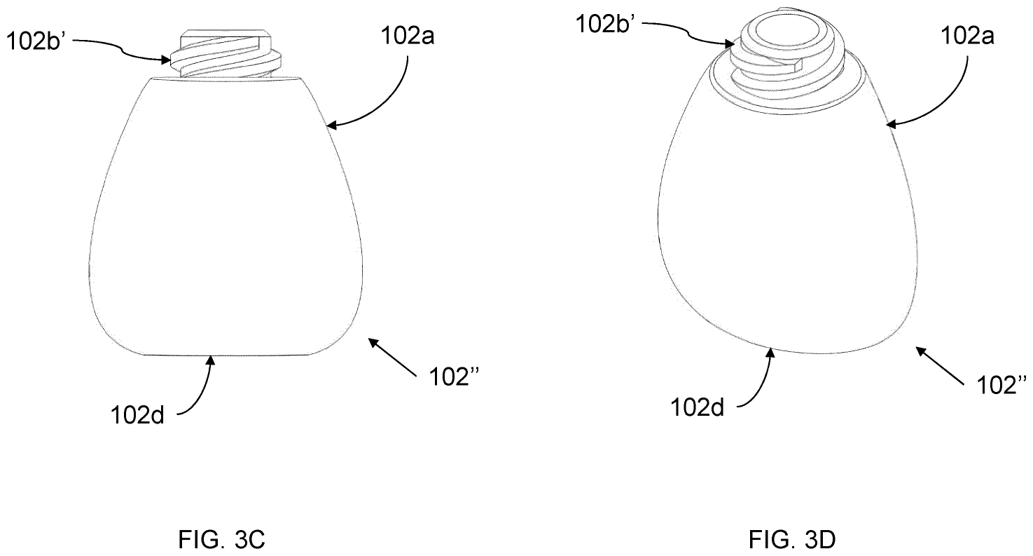

While the pipette element 102 of the embodiment shown in FIGS. 1 and 2 comprises a male ENFit connector 102b, in alternative embodiments the pipette element 102 may comprise a female ENFit connector hub. FIG. 3A shows the pipette element 102 comprising a male ENFit connector 102b in more detail, while FIG. 3B shows a pipette element 102′ comprising a female ENFit connector hub 102c. In some embodiments, the ENFit connector of the pipette element 102 may comprise extended threading. This threading is compatible with the ENFit standard, and may allow the connection with the capping part 109 to be more effectively secured. An example of such extended threading is illustrated in FIGS. 3C and 3D, which show a pipette element 102″ comprising a male ENFit connector 102b′ with extended threading. In embodiments in which a pipette element 102″ having a connector 102b′ comprising extended threading is used, the capping part 109 is configured to receive the ENFit connector 102b′ including the extended threading. It will be appreciated however that a variety of other connector types may be used. For example, the pipette element 102 may be connected to capping part 109 using a snap-on connector or a friction connector. In some embodiments the pipette element 102 and the capping part 109 may be molded into a single piece, e.g. with a frangible connection, which is broken when the pipette element 102 is removed.

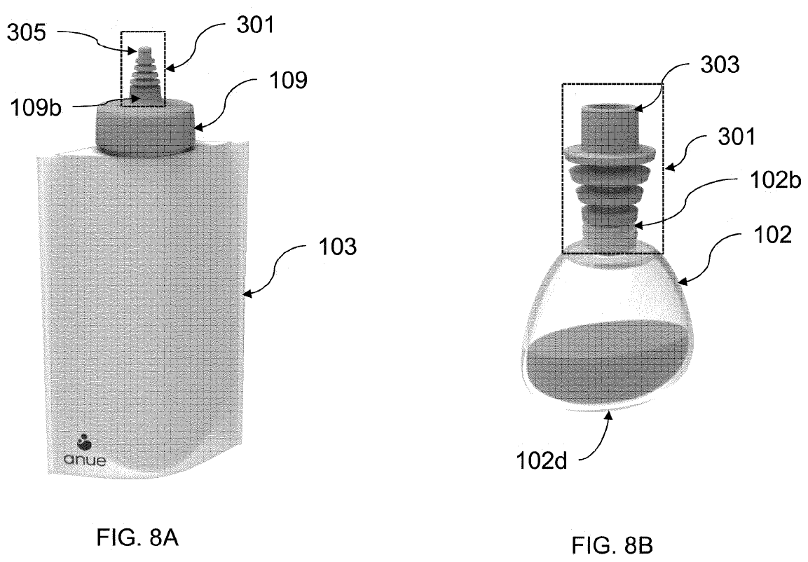

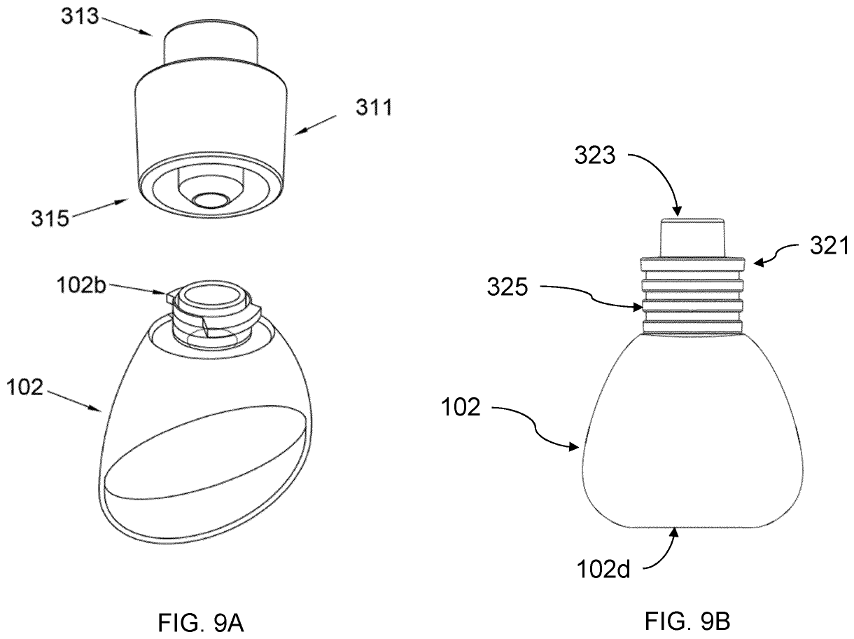

In the embodiments seen in FIGS. 1-3, the pipette element 102 includes a flat base portion 102d configured to support the pipette element 102 in an upright standing position. This means that the pipette element 102 containing the liquid sample (in liquid or frozen state) can conveniently stand on a surface e.g. when testing the sample. This is described further in relation to FIGS. 8B and 9B.

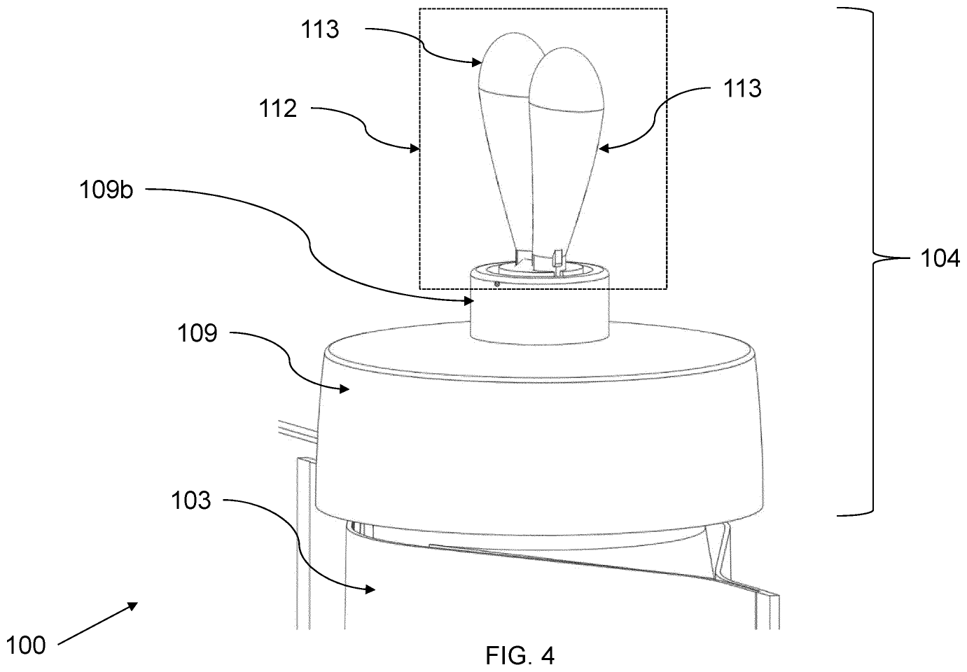

FIG. 4 shows a dual pipette element 112 which may be used with the storage vessel 101 in place of the pipette element 102 in some embodiments of the invention. The dual pipette element 112 comprises two pipette chambers 113 which can be attached to the capping part 109 described previously. The dual pipette element 112 may be used when two individual samples of a liquid need to be collected for different purposes. For example, the dual pipette element 112 may be used to collect two samples of breastmilk from the storage vessel 101: a first sample for bacteriological testing, and a second sample for testing the nutritional contents of the breastmilk.

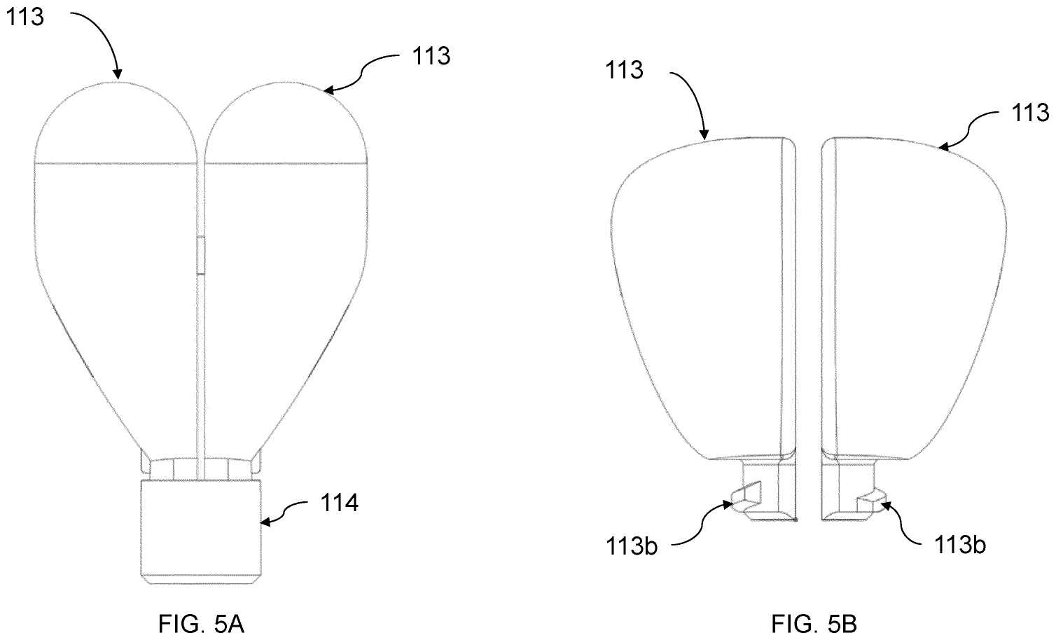

FIG. 5 shows two possible types of connection between the dual pipette element 112 and the capping part 109. FIG. 5A shows two pipette chambers 113 of the dual pipette element 112 held together in an adapter 114 by friction. The adapter 114 may be any suitable adapter configured to mate with the connector 109b of the capping part 109. The pipette chambers 113 may be removed from storage vessel 101 inside the adapter 114 as a pair, and may later be separated and removed individually from the adapter. Alternatively, the pipette chambers 113 may be removed individually while the adapter 114 is in-situ. FIG. 5B shows an alternative embodiment in which each of the pipette chambers 113 terminates in a connector equivalent to half of an ENFit connector, hereinafter referred to as a half-connector 113b. A connection may be formed between a female ENFit connector hub 109b on the capping part 109, and a male ENFit connector formed by the combination of the two half-connectors 113b on each of the pipette chambers 113 when the pipette chambers 113 are placed next to one another. In some embodiments the pipette chambers 113 may be snap-locked to one another in order to facilitate their use.

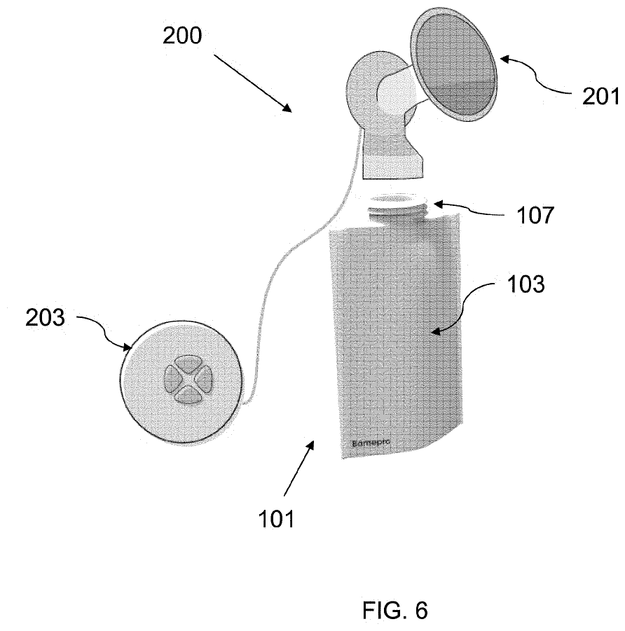

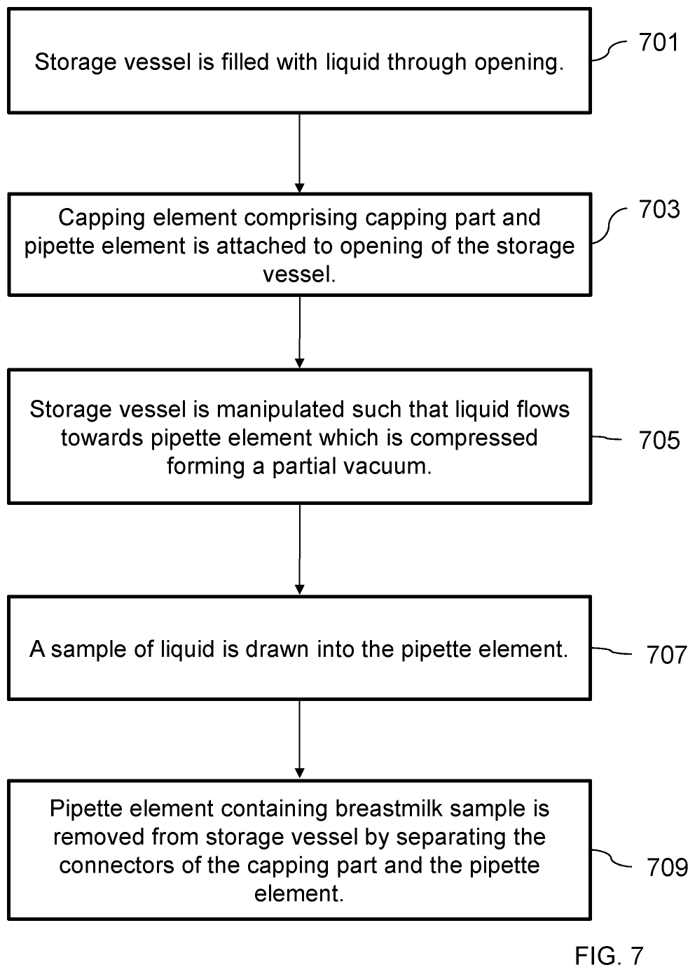

The storage vessel 101 may be connected to additional components at the screw thread interface 107 of the opening 105 or at the connector 109b of the capping part 109. FIG. 6 shows a breast pump set 200 used with the storage vessel 101 of FIG. 1. The breast pump set 200 includes a breast shield 201 connected to the pouch 103 via the screw thread 107. The breast shield 201 is connected to a breast pump 203 (electric or manual) such that breastmilk can be expressed directly into the pouch 103, without requiring the use of additional containers, such as the collection bottle of a breast pump, reducing the risk of contamination of the breastmilk. Once pumping is complete and the pump set 200 has been removed, other components may be connected at the screw thread 107 of the opening 105, for example a capping element 104 as already described above. Once a sample has been collected in the pipette element 102, and the pipette element 102 removed, an enteral feeding syringe may be connected to the connector 109b of the remaining capping part 109 in order to transfer breastmilk directly from the pouch 103 to a prematurely born baby. The potential for contamination is therefore minimized. These steps may all take place while the breastmilk is fresh, e.g. if it is expressed in a hospital, or the breastmilk may be frozen for later use, as described further below.

FIG. 7 is a flow chart showing the process of collecting a sample of breastmilk using embodiments of the liquid sample collection system 100 including a pipette element 102 as described in FIGS. 1-6. In step 701, the pouch 103 of the storage vessel 101 is at least partially filled with breastmilk. Breastmilk may be expressed directly into the storage vessel 101 as explained in relation to FIG. 6, or may be decanted into the storage vessel 101 from another container, such as the collection bottle of a breast pump. In step 703, the capping element 104 is attached to the storage vessel 101 at the opening 105. In step 705, the storage vessel 101 is turned such that the breastmilk within the pouch 103 flows towards the pipette element 102, which is compressed, forming a partial vacuum inside the bulb 102a of the pipette element 102. In step 707, the pressure on the bulb 102a is released, and a sample of breastmilk from the storage vessel 101 is drawn into the bulb 102a of the pipette element 102 by the partial vacuum, where it is held in place by the surface tension of the breastmilk, as a sample separated from the liquid in the storage vessel 101. Thus, the sample of breastmilk contained within the pipette element 102 is no longer in fluid contact with the liquid in the pouch 103, and does not flow back into the pouch 103 in the event that the storage vessel 101 is turned such that the pipette element is above the pouch 103. Prior to drawing a sample into the pipette element 102, the storage vessel 101 may be shaken in order to ensure that a homogenous and representative sample is collected in the pipette element 102. In step 709, the pipette element 102 containing the sample of breastmilk is removed from the storage vessel 101, and can be used to transfer a small sample of breastmilk for testing. The storage vessel 101 and attached capping element 104 (including the capping part 109 and pipette element 102) may be frozen before the pipette element 102 is removed. Step 709 may therefore take place following transportation of the collection system 100, e.g. to a hospital milk bank.

After removing the pipette element 102 from the storage vessel 101, the remaining breastmilk stored therein may be protected from contamination using a plug 301. An example of a plug 301 suitable for embodiments in which a friction connector is used is shown in FIGS. 8A and 8B. In this embodiment the plug 301 has a generally conical shape, with a cylindrical projection 303 at one end and a tapered tip 305 at the opposite end. The plug 301 is configured to seal both the pouch 103 (by sealing the connector 109b of the capping part 109) and the pipette element 102, either together or individually. The cylindrical projection 303 from the base of the plug 301 is configured to mate with the connector 109b of the capping part 109, and the tip 305 of the plug 301 is configured to mate with the connector 102b of the pipette element 102. As can be seen in FIG. 8B, the plug 301 may be used to seal the opening of the pipette element 102 while the pipette element 102 rests on the flat base portion 102d. In this way the likelihood of contamination of a sample of liquid stored in the pipette element 102 may be reduced while it is being further transported or while awaiting testing of the liquid sample.

FIGS. 8A and 8B show the plug 301 being used to seal the pouch 103 (by sealing the connector 109b of the attached capping part 109) and the pipette element 102 respectively. Although FIGS. 8A and 8B show a plug 301 configured to seal the pouch 103 and the pipette element 102 using a friction connector, it will be appreciated that the cylindrical projection 303 and tapered tip 305 of the plug 301 may be configured to mate with a range of connector types, e.g. ENFit connectors or snap-on connectors.

In other embodiments a plug 311 with a generally cylindrical shape may be used, as shown in FIGS. 9A and 9B. FIG. 9A shows a plug 311 with a generally cylindrical shape and configured to seal a storage vessel (by sealing the connector 109b of the attached capping part 109 as seen in FIG. 8A) and the pipette element 102. The plug 311 comprises a narrow section 313 configured to mate with the connector 109b of the capping part 109, and a wide section 315 configured to mate with the connector 102b of the pipette element 102. In the embodiment shown in FIG. 9A, the plug 311 is made of a compressible material such as rubber or silicone, such that it can form a seal around the connector 102b of the pipette element 102. While in FIG. 9A the plug 311 is shown as having a smooth surface, in other embodiments, a plug with a ribbed surface may be used. FIG. 9B shows a plug 321 according to another embodiment of the invention. The plug 321 has a ribbed surface, and comprises a narrow section 323 configured to mate with the connector 109b of the capping part 109, and a wide section 325 configured to mate with the connector 102b of the pipette element 102. The ribbed surface of the plug 321 may advantageously allow the plug 321 to be more easily gripped by a user when used to seal/unseal the capping part 109 or the pipette element 102.

Although FIGS. 9A and 9B show plugs 311, 321 configured to seal a storage vessel and a pipette element using an ENFit connector, it will be appreciated that the plugs 311, 321 may be configured to mate with a range of connector types, e.g. friction connectors or snap-on connectors.

In some embodiments according to the first aspect of the invention, the capping element 104 does not comprise a pipette element 102. Instead, it will be appreciated that a range of alternative caps comprising a chamber capable of storing a sample of liquid may be used.

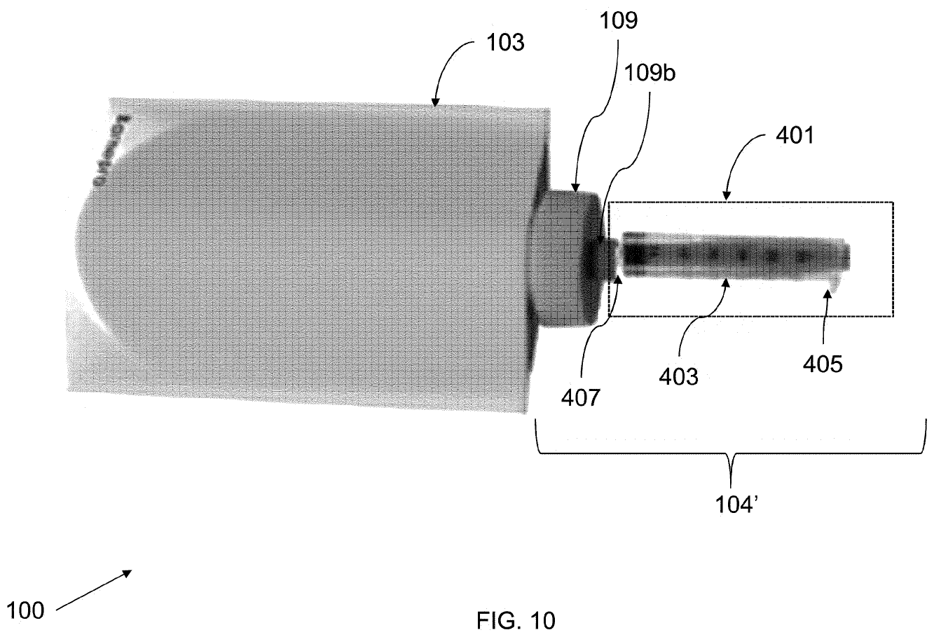

FIG. 10 shows an embodiment of a liquid sample collection system 100 according to the first aspect of the invention, in which the pipette element 102 is replaced by a syringe element 401 in a capping element 104′ comprising the syringe element 401 and a capping part 109. As can be seen in FIG. 10, the syringe element 401 is connected to a storage pouch 103 of a storage vessel 101 via the capping part 109. The capping part 109 and the pouch 103 are identical to those described in relation to FIGS. 1-7 and the description is not repeated here.

The syringe element 401 has a barrel 403, a plunger 405, and a connector 407 configured to mate with the connector 109b of the capping part 109. The connector 407 may be any appropriate connector configured to mate with the connector 109b of the capping part 109. In some embodiments the connector 407 may comprise an ENFit connector. In some embodiments, the connector 407 may comprise a snap-on connector or a friction connector. In some embodiments syringe element 401 and capping part 109 may be molded into a single piece, which is broken when the syringe element 401 is removed.

The syringe element 401 is used in a similar manner to the pipette element 102 as described in relation to FIG. 7. The collection of a sample using the syringe element 401 differs from that of the pipette element 102 in that a partial vacuum is formed within the barrel 403 of the syringe element 401 rather than within the bulb 102a of the pipette element 102. The connector 407 of the syringe element 401 is attached to the connector 109b of the capping element 109 with the plunger 405 fully inserted within the barrel 403. The storage vessel 101 is then turned such that its contents flow towards the syringe element 401, and the plunger 405 is partially withdrawn from the barrel 403. This forms a partial vacuum within the barrel 403 of the syringe element 401, causing liquid from within the storage vessel 101 to flow into the barrel 403 of the syringe element 401, where it is held separately to the liquid remaining in the storage vessel 101. Once a sample has been drawn into the barrel 403 of the syringe element 401, the syringe element 401 containing the sample of breastmilk is disconnected from the storage vessel 101, and can be used to transfer a small sample of breastmilk, e.g. to an appropriate container for testing. The storage vessel 101 and attached syringe element 401 may be frozen before the syringe element 401 is removed in order to preserve the quality of the sample within the syringe element 401 and prevent possible contamination. After the syringe element 401 is removed, the storage vessel 101 and the syringe element 401 may be sealed with a plug as described in relation to FIGS. 8A, 8B, 9A and 9B, with the pipette element 102 replaced by the syringe element 401 which has an equivalent connector.

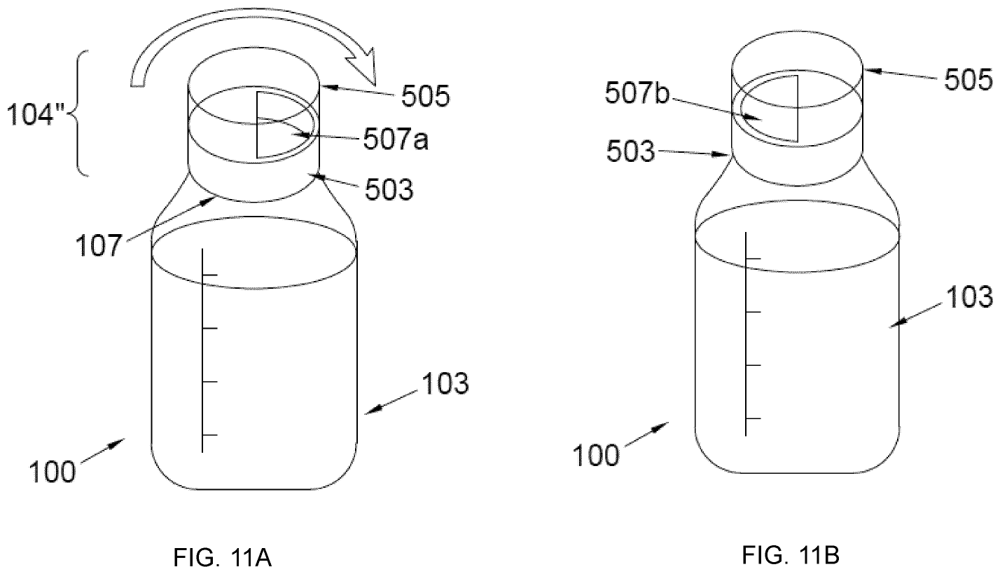

FIGS. 11A and 11B show a liquid sample collection system 100 according to a further embodiment of the first aspect of the invention, in which a capping element 104″ comprising a chamber 505 and a capping part 503 is used. The capping element 104″ is connected to the opening 105 of the pouch 103 by the screw thread interface 107, and in the embodiment shown comprises two parts: a capping part 503, and a chamber 505. The capping part 503 and the chamber 505 comprise first and second apertures 507a and 507b respectively. The chamber 505 is rotatable around a central axis of the opening 105 such that the second aperture 507b can be rotated between a first position in which it is in fluid communication with the first aperture 507a (shown in FIG. 11A), and a second position in which there is no fluid communication between the first aperture 507a and the second aperture 507b (shown in FIG. 11B).

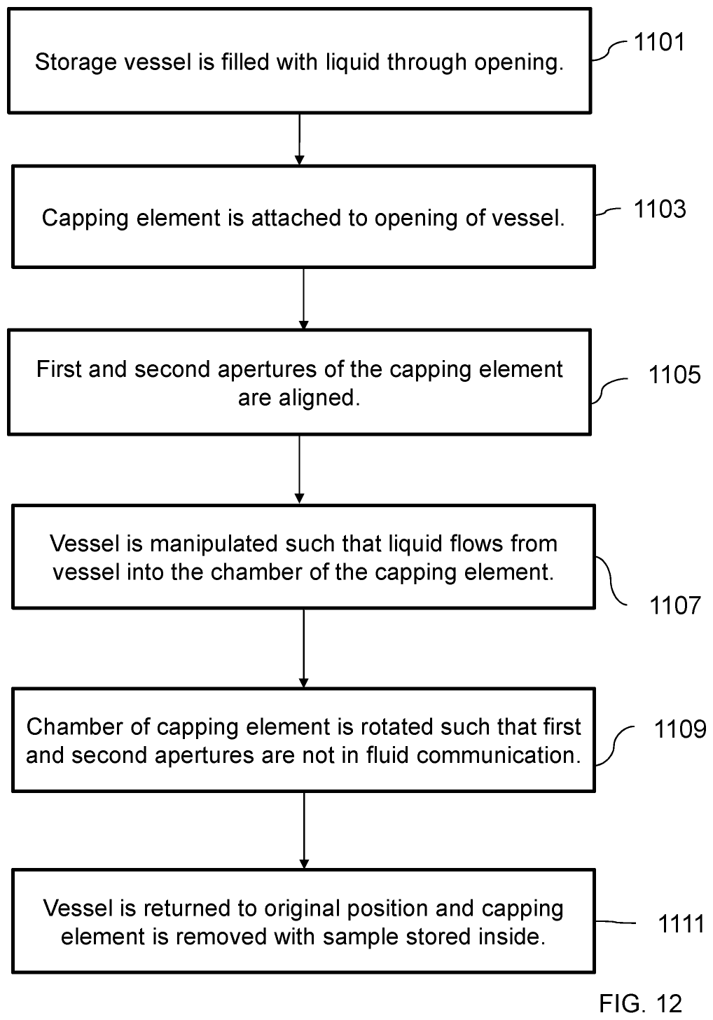

The capping element 104″ may be used to collect a sample of breastmilk from the pouch 103 as will be described in relation to FIG. 12. FIG. 12 is a flow chart showing the process of collecting a sample of breastmilk using embodiments of the liquid sample collection system 100 including a capping element 104″ as shown FIG. 11.

In step 1101, the pouch 103 of storage vessel 101 is partially filled with breastmilk. Breastmilk may be expressed directly into the pouch 103 of the storage vessel 101 as explained in relation to FIG. 6, or may be decanted into the pouch 103 from another container, such as the collection bottle of a breast pump. In step 1103, the capping element 104″ is attached to the pouch 103 at the opening 105. In step 1105 the chamber 505 is rotated with respect to the capping part 503 such that the second opening 507b comes into fluid communication with the first aperture 507a, allowing breastmilk to flow between the storage vessel 101 and the chamber 505. In step 1107, the storage vessel 101 is turned such that the breastmilk within the vessel flows towards the capping element 104″, such that breastmilk flows into the chamber 505. In step 1109, the chamber 505 is rotated with respect to the capping element 503 such that the second aperture 507b is no longer in fluid communication with the first aperture 507a, sealing a sample of liquid within the chamber 505. In step 1111, the capping element 104″ containing the sample of breastmilk within the chamber 505 is removed from the storage vessel 101, and can be used to transfer a small sample of breastmilk to an appropriate container for testing. The storage vessel 101 and attached capping element 104″ may be frozen before the capping element 104″ is removed in order to preserve the quality of the sample within the second capping element 104″ and prevent possible contamination of the sample.

In some embodiments the capping element 104″may comprise a chamber 505 and a cover which may be rotated between a position in which the opening 105 of the pouch 103 is fully covered, preventing the flow of liquid into the capping element 104″, and a second position in which the opening 105 of the pouch 103 is at least partially open, allowing the flow of liquid into the capping element 104″.

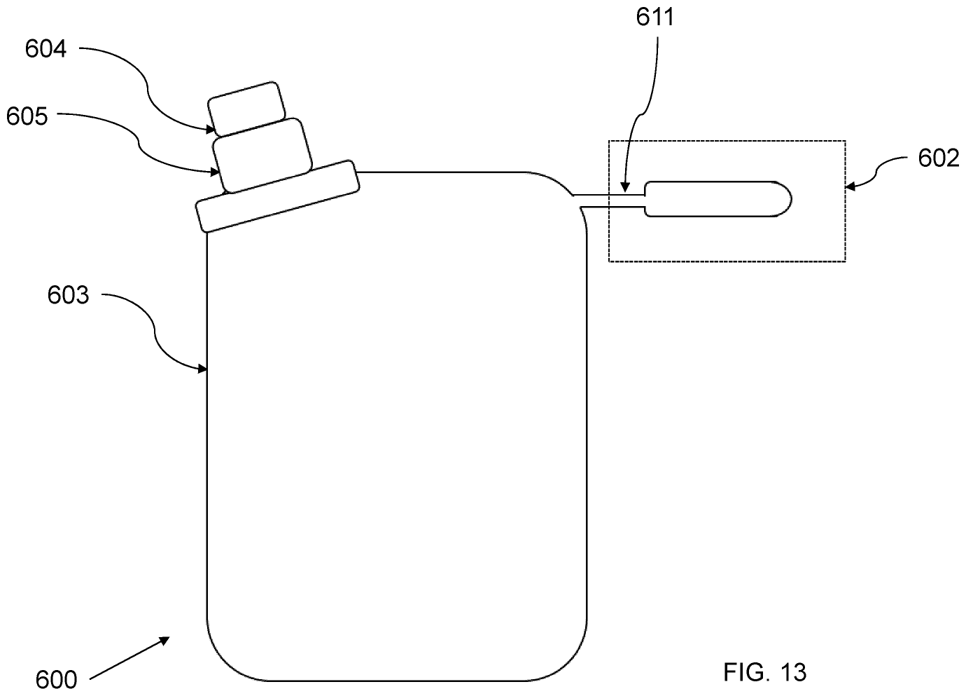

In accordance with another aspect of the invention, samples of liquid may be collected from a pouch 603 without the use of capping elements configured to store a sample of liquid. FIG. 13 shows a liquid sample collection system 600 consisting of a storage vessel 601 comprising a pouch 603, an opening 605, a cap 604, and a pipette element 602 formed integrally with the pouch 603. The pouch 603 and the pipette element 602 may be formed of a thermoplastic material, and are preferably transparent such that their contents can be easily observed. The pipette element 602 is connected to the pouch 603 by the pipette body 611. The chamber of the pipette element 602 may have an internal volume of up to 20 ml, but in preferred embodiments has a volume of 2-5 ml. The opening 605 may comprise a screw thread 607 (not shown), to which the cap 604 can be connected with a corresponding threading on its interior surface. The screw thread 607 of the opening 605 can also be used to connect additional components such as a single use disposable breast shield connected to a breast pump in a comparable manner to that described in relation to the embodiment shown in FIG. 6. In this way breastmilk can be expressed directly into the pouch 603 without requiring the use of additional containers, such as the collection bottle of a breast pump, reducing the risk of contamination of the breastmilk.

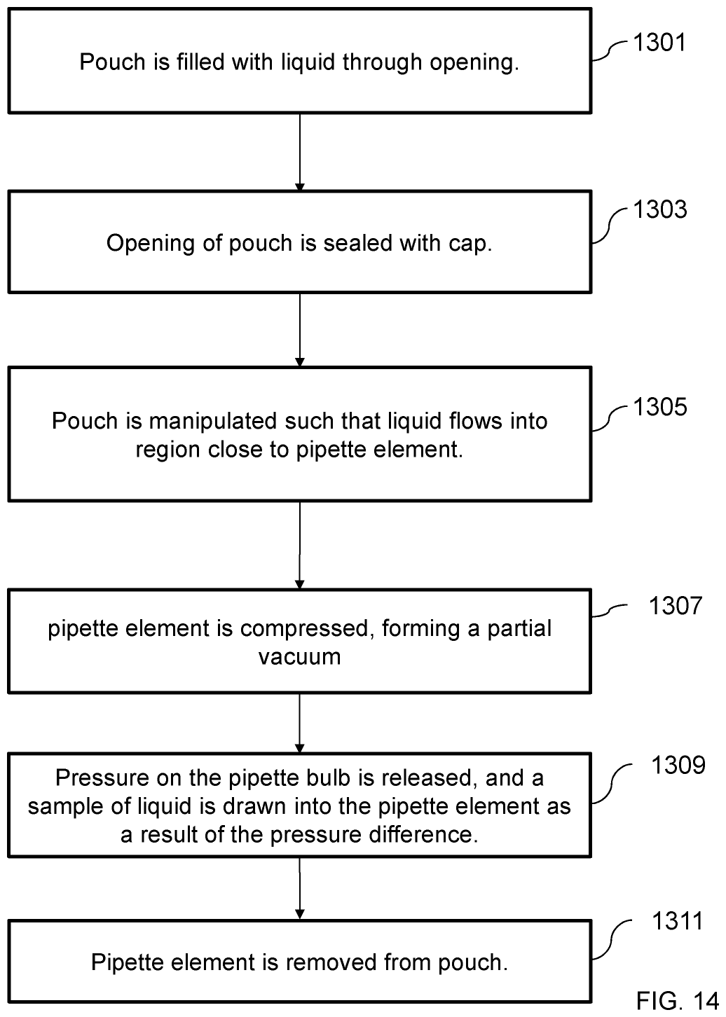

FIG. 14 is a flow chart showing the process of collecting a sample of breastmilk using embodiments of the liquid sample collection system 100 including a pouch 603 comprising an integrated pipette element 602. In step 1301, the pouch 603 of is partially filled with breastmilk. Breastmilk may be expressed directly into the pouch 603, or may be decanted into the pouch 603 from another container, such as the collection bottle of a breast pump. In step 1303, the opening 605 of the pouch 603 is sealed using the cap 604. In step 1305 the pouch 603 is manipulated such that liquid flows liquid flows into region close to pipette element. In step 1307 the pipette element 602 is compressed, forming a partial vacuum inside the bulb of the pipette element 602. In step 1309, the pressure on the bulb of the pipette element 602 is released, and a sample of breastmilk from the pouch 603 is drawn into the bulb of the pipette element 602 through the pipette body 611 by the partial vacuum, and is held in place within the bulb of the pipette element 602 by the surface tension of the breastmilk. Alternatively, the pipette body 611 can be sealed using heat sealing, such as using a hot bar welding or impulse welding process, in which opposite sides of the pouch and of the pipette element are fused together by the application of heat to the thermoplastic material.

Prior to drawing a sample into the pipette element 602, the sealed pouch 603 may be shaken in order to ensure that a homogenous and representative sample is collected in the pipette element 602. In step 1311, the pipette element 102 containing the sample of breastmilk is removed from the pouch 603. This may be achieved using a heat weld as described above. After the pipette element 602 has been removed, it can be used to transfer a small sample of breastmilk to an appropriate container for testing. As the pipette element 602 is sealed by the heat weld process, an opening must be made in the pipette element 602 to remove the sample. The pouch 603 and attached pipette element 602 may be frozen before the pipette element 602 is removed in order to preserve the quality of the sample within the pipette element 602 and prevent possible contamination.

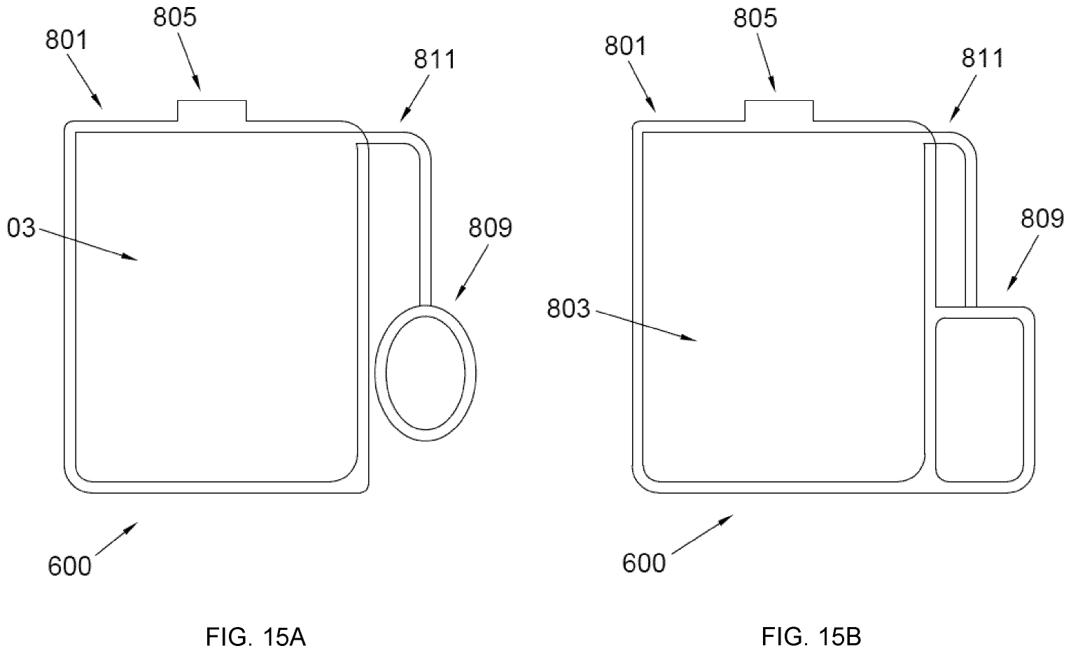

FIGS. 15A and 15B show further embodiments of the liquid sample collection system 600 consisting of a storage vessel 801 in which a sample of liquid may be collected from a pouch 803 without the use of a capping element configured to store a sample of liquid.

FIG. 15A shows a pouch 803 with an opening 805 and a compartment 809 connected to the pouch by a channel 811. The compartment 809 and the pouch 803 may be made of the same thermoplastic material, and are both preferably transparent such that their contents can be easily observed. The channel 811 is configured to allow liquid to flow between the pouch 803 and the compartment 809. The channel 811 may be made of the same material or may be made of a different material such as polyvinylchloride (PVC) or polyurethane. In some embodiments the channel comprises tubing. The opening 805 of the pouch 803 may be connected to a cap 804 (not shown), which may comprise a screw thread 807 to which the cap 604 can be connected with a corresponding threading on its interior surface. The screw thread 807 of the opening 805 can also be used to connect additional components such as a single use disposable breast shield connected to a breast pump in a comparable manner to that described in relation to the embodiment shown in FIG. 6. In this way breastmilk can be expressed directly into the pouch 803 without requiring the use of additional containers, such as the collection bottle of a breast pump, reducing the risk of contamination of the breastmilk.

FIG. 15A shows an embodiment in which the pouch 803 and the compartment 809 are separate and joined only by the channel 811, while FIG. 15B shows an embodiment in which the compartment 809 and the pouch 803 are connected together.

The liquid sample collection system 600 shown in FIGS. 15A and 15B may be used to collect a small sample of liquid in the second compartment separately from the larger volume stored within the pouch 803. The pouch 803 may first be manipulated such that a sample of liquid flows through the channel 811 and into the compartment 809. The pouch 803 may then be turned back to a position in which the liquid stored in the pouch 803 and the liquid stored in the compartment 809 are separate, i.e. so that there is minimal, or no liquid present in the channel 811. The channel may 811 then be sealed using a heat weld. The compartment 809 (and optionally the channel 811) may also be removed from the pouch 803 using a heat weld, sealing a sample of liquid within the removed compartment 809. As in previously described embodiments, the liquid sample collection system 600 may be frozen before the compartment 809 is removed in order to preserve the quality of the sample within the compartment 809 and prevent possible contamination.

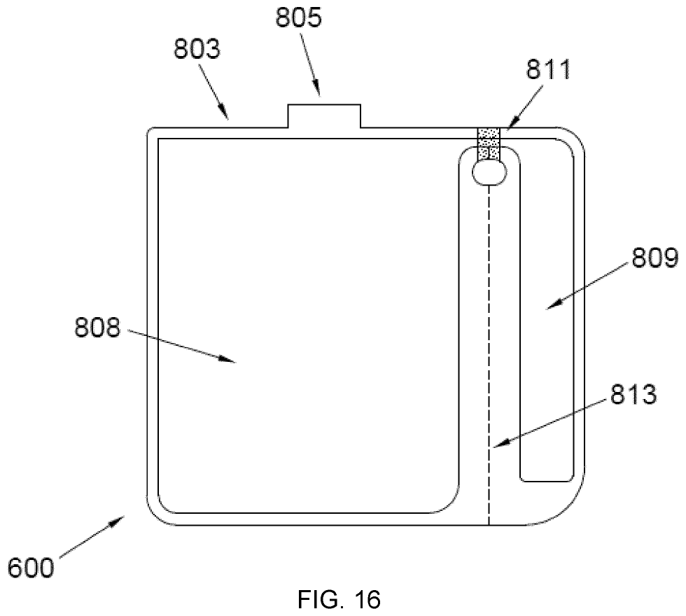

In some embodiments the channel 811 may be integral with the pouch 803. FIG. 16 shows an embodiment in which the pouch 803 comprises a first compartment 808 and a second compartment 809, joined by a by a narrow channel 811. Forming the first and second compartments within the same thermoplastic structure may significantly reduce the manufacturing cost of the liquid sample collection system 600. The liquid sample collection system 600 shown in FIG. 16 also comprises a perforated line 813 between the first compartment 808 and the second compartment 809 in order to facilitate the disconnection of the channel 811, and the removal of the second compartment 809 containing the liquid sample. In the embodiment shown in FIG. 16, a heat weld only needs to be applied to the narrow channel 811 to remove the second compartment 809 containing the liquid sample.

It will be appreciated by those skilled in the art that the disclosure has been illustrated by describing one or more specific examples thereof, but is not limited to these examples; many variations and modifications are possible, within the scope of the accompanying claims.

What is claimed is:

1-35. (canceled)

36. A system for collecting a sample of a liquid, the system comprising:
a liquid storage vessel comprising an opening; and
a capping element configured to seal the opening of the storage vessel;
wherein the capping element comprises:
a pipette element configured to store a sample of the liquid separate to the liquid storage vessel; and
a capping part connectable between the opening and the pipette element;
wherein the pipette element is separable from the capping part;
wherein the capping part comprises a first liquid conveying connector;
wherein the pipette element comprises a second liquid-conveying connector configured to mate with the first liquid-conveying connector; and
wherein the first liquid conveying connector is uncovered when the pipette element is separated from the capping part.

37. The system of claim 36, wherein the pipette element comprises a flat base portion configured to support the pipette element in an upright standing position.

38. The system of claim 36, wherein the capping element comprises a dual pipette element defining two pipette chambers,
wherein each pipette chamber is configured to store some of the sample of liquid taken from the storage vessel.

39. The system of claim 36, wherein the two pipette chambers are separable from one another.

40. The system of claim 36, wherein the first liquid-conveying connector comprises an ENFit connector.

41. The system of claim 36, wherein the second liquid-conveying connector comprises an ENFit connector.

42. The system of claim 36, further comprising a plug;
wherein the plug comprises two ends: a first end configured to seal the first liquid conveying connector of the capping part, and a second end configured to seal the second liquid conveying connector of the pipette element after the pipette element is separated from the capping part

43. The system of claim 36, wherein the opening of the liquid storage vessel comprises a threaded interface.

44. The system of claim 36, wherein the opening of the storage vessel is effectively sealed by the capping element.

45. The system of claim 36, wherein the second liquid-conveying connector comprises a screw fitting.

46. The system of claim 36, wherein the liquid storage vessel comprises a pouch or a bottle.

47. The system of claim 36, wherein the pipette element is at least partially transparent.

48. A method of collecting a sample of liquid from a storage vessel, the method comprising:
at least partially filling the storage vessel with a liquid;
sealing the storage vessel with a capping element; the capping element comprising:
a pipette element configured to store a sample of the liquid separate to the liquid storage vessel; and
a capping part connectable between the opening and the pipette element;
the method further comprising:
manipulating the storage vessel and/or the capping element such that a sample of the liquid flows from the storage vessel into the pipette element of the capping element; and
storing the sample of liquid in the pipette element of the capping element.

49. The method of claim 48, wherein the liquid is breastmilk, and wherein the method comprises:
connecting a breast pump to the storage vessel and operating the breast pump to introduce breastmilk into the storage vessel.

50. The method of claim 48, further comprising removing the pipette element containing the sample of liquid.

51. The method of claim 50, wherein removing the pipette element comprises removing the capping element from the storage vessel, and optionally resealing the storage vessel.

52. The method of claim 48, further comprising the steps of:
freezing the storage vessel and the capping element containing the sample of liquid; and
removing the pipette element containing the frozen sample.

53. The method of claim 52, wherein removing the pipette element comprises separating the pipette element from the capping part.

54. The method of claim 53, wherein separating the pipette element from the capping part comprises uncovering a first liquid conveying connector of the capping part.

55. The method of claim 53, further comprising sealing the capping part or the pipette element.
